(19) United States
(12) Patent Application Publication
(10) Pub. No.: US 2023/0206302 A1
(43) Pub. Date: Jun. 29, 2023

(54) INFORMATION PROCESSING METHOD, INFORMATION PROCESSING DEVICE, AND NON-TRANSITORY COMPUTER READABLE RECORDING MEDIUM STORING INFORMATION PROCESSING PROGRAM

(71) Applicant: Panasonic Intellectual Property Corporation of America, Torrance, CA (US)

(72) Inventors: Toshiaki TANAKA, Hyogo (JP); Kana MIYATA, Osaka (JP); Yuki MINODA, Hyogo (JP); Ken OHMAE, Tokyo (JP); Yushi NAKAMURA, Osaka (JP); Koh UEHARA, Osaka (JP)

(73) Assignee: Panasonic Intellectual Property Corporation of America, Torrance, CA (US)

(21) Appl. No.: 18/112,925

(22) Filed: Feb. 22, 2023

Related U.S. Application Data
(63) Continuation of application No. PCT/JP2021/030867, filed on Aug. 23, 2021.
(60) Provisional application No. 63/072,344, filed on Aug. 31, 2020.

(30) Foreign Application Priority Data
Aug. 4, 2021 (JP) 2021-128551

(51) Int. Cl. G06Q 30/0601; A61B 5/01

(52) U.S. Cl. CPC G06Q 30/0631; A61B 5/01

(57) ABSTRACT

A body temperature management server executes: acquiring body temperature information indicating a body temperature of a user and associated with a user ID specifying the user; acquiring external information generated on the basis of preference information about the user; generating, on the basis of the body temperature information and the external information, recommendatory information for the user specified by the user ID; and outputting the generated recommendatory information.

### TECHNICAL FIELD

This disclosure relates to a technology of generating, on the basis of body temperature information about a user, recommendatory information recommendable for the user.

### BACKGROUND ART

For instance, Patent Literature 1 discloses a system for monitoring health of an employee while the employee is located in an employee workstation. The system disclosed in Patent Literature 1 executes: collecting health data including neural data collected from one or more neural sensors disposed about a scalp of the employee; determining, on the basis of the neural data, an employee health profile; generating, on the basis of the employee health profile, a health plan for the employee; and supplying a health content including the employee health profile and the health plan for displaying to the employee.

However, the conventional technology fails to consider providing recommendatory information attracting a user to have an interest over an entirety of life as well as a healthy action of an employee in a workplace, and thus needs further improvement.

### CITATION LIST

### Patent Literature

Patent Literature 1: Japanese Unexamined Patent Publication No. 2019-57301

### SUMMARY OF INVENTION

This disclosure has been achieved to solve the drawbacks described above, and has an object of providing a technology for providing recommendatory information attracting a user to have an interest over an entirety of life from body temperature information about the user.

An information processing method according to this disclosure, by a computer, includes: acquiring body temperature information indicating a body temperature of a user and associated with a user ID specifying the user; acquiring external information generated on the basis of preference information about the user; generating, on the basis of the body temperature information and the external information, recommendatory information for the user specified by the user ID; and outputting the recommendatory information.

This disclosure achieves provision of recommendatory information attracting a user to have an interest over an entirety of life from body temperature information about the user.

### BRIEF DESCRIPTION OF DRAWINGS

FIG. 1 is a diagram showing an example of a configuration of a body temperature management system in a first embodiment of the disclosure.

FIG. 2 is a flowchart explaining notification of information by a body temperature management server in the first embodiment of the disclosure.

FIG. 3 shows an example of a recommendatory information presentation screen image displayed on a user terminal in the first embodiment.

FIG. 4 is a diagram showing an example of a configuration of a body temperature management system in a second embodiment of the disclosure.

FIG. 5 is a first flowchart explaining notification of information by a body temperature management server in the second embodiment of the disclosure.

FIG. 6 is a second flowchart explaining the notification of information by the body temperature management server in the second embodiment of the disclosure.

FIG. 7 is a diagram showing an example of a configuration of a body temperature management system in a third embodiment of the disclosure.

FIG. 8 is a flowchart explaining notification of information by a body temperature management server in the third embodiment of the disclosure.

FIG. 9 shows an example of a recommendatory information presentation screen image displayed on a user terminal in the third embodiment.

FIG. 10 is a diagram showing an example of a configuration of a body temperature management system in a fourth embodiment of the disclosure.

FIG. 11 is a flowchart explaining notification of information by a body temperature management server in the fourth embodiment of the disclosure.

FIG. 12 shows an example of a recommendatory information presentation screen image displayed on a user terminal in the fourth embodiment.

### DESCRIPTION OF EMBODIMENTS

Knowledge forming the basis of the present disclosure

In the aforementioned conventional technology, a health content to be provided to an employee is generated only on the basis of a sensing result about the employee in a workplace. For instance, examples of the health content include an eye information dialog for providing the employee with a suggestion of reducing eye fatigue. The dialog gives the suggestion of recommending the employee to take a break away from a computer every twenty minutes and stare at a certain object twenty feet away for one minute in the break time.

It is seen from this perspective that a matter in the health content to be provided to the employee is limited to a healthy action of the employee in the workplace in the conventional technology. Hence, the conventional technology fails to consider providing recommendatory information attracting a user to have an interest over an entirety of life as well as the healthy action of the employee in the workplace.

To solve the above-described drawbacks, an information processing method according to one aspect of this disclosure, by a computer, includes: acquiring body temperature information indicating a body temperature of a user and associated with a user ID specifying the user; acquiring external information generated on the basis of preference information about the user; generating, on the basis of the body temperature information and the external information, recommendatory information for the user specified by the user ID; and outputting the recommendatory information.

According to this configuration, recommendatory information is generated for the user on the basis of body temperature information indicating a body temperature of the user and external information generated on the basis of preference information about the user, and the generated recommendatory information is output. This achieves provision of recommendatory information attracting the user to have an interest over an entirety of life from the body temperature information about the user.

In the information processing method, the external information may include a set of restaurant information about restaurants recommendable for the user. In the generating of the recommendatory information, when the body temperature information indicates a predetermined value or lower, the recommendatory information including the set of restaurant information may be generated.

According to this configuration, when the body temperature information about the user indicates the predetermined value or lower, recommendatory information including a set of restaurant information about restaurants recommendable for the user is generated. Therefore, for example, a restaurant in which the user has an interest is representable when the user has no fever.

The information processing method may further include acquiring vacancy information about vacancy availability of each of the restaurants recommendable for the user. In the generating of the recommendatory information, when the body temperature information indicates the predetermined value or lower, restaurant information about a restaurant having a vacancy may be selected, on the basis of the vacancy information, from the set of restaurant information, and the recommendatory information including the selected restaurant information may be generated.

According to this configuration, when body temperature information about the user indicates the predetermined value or lower, restaurant information about a restaurant having a vacancy is selected on the basis of vacancy information indicating vacancy availability of each restaurant recommendable for the user from the set of restaurant information recommendable for the user, and recommendatory information including the selected restaurant information is generated. Therefore, for example, a restaurant in which the user has an interest and vacancy availability or nonavailability of the restaurant are presentable when the user has no fever.

The information processing method may further include acquiring congestion density information about a congestion density in a region where each of the restaurants recommendable for the user is located. In the generating of the recommendatory information, when the body temperature information indicates the predetermined value or lower, restaurant information about a restaurant located in a region whose congestion density indicates a predetermined value or smaller may be selected from the set of restaurant information, and the recommendatory information including the selected restaurant information may be generated.

According to this configuration, when the body temperature information about the user indicates the predetermined value or lower, restaurant information about a restaurant located in a region whose congestion density indicates a predetermined value or lower is selected from the set of restaurant information recommendable for the user, and recommendatory information including the selected restaurant information is generated. Therefore, for example, a restaurant which is located in a region whose congestion density indicates the predetermined value or lower and in which the user has an interest is representable when the user has no fever.

In the information processing method, the external information may include a set of recipe information about dish recipes recommendable for the user. In the generating of the recommendatory information, recipe information may be selected, on the basis of the body temperature information, from the set of recipe information, and the recommendatory information including the selected recipe information may be generated.

According to this configuration, recipe information is selected, on the basis of body temperature information, from a set of recipe information recommendable for the user, and the recommendatory information including the selected recipe information is generated. Consequently, a dish recipe which is optimal to a current body temperature of the user and attractive to the user to have an interest is presentable.

In the information processing method, the external information may include preset bathing temperature information about a temperature of warm water to be placed in a bath tab, the temperature being preset by the user. In the generating of the recommendatory information, a specific temperature of warm water to be placed in the bath tab may be determined on the basis of the body temperature information, and the recommendatory information including bathing temperature information indicating the determined temperature may be generated when the determined temperature differs from the preset bathing temperature information.

According to this configuration, a specific temperature of warm water to be placed in the bath tab is determined on the basis of a body temperature of the user. Moreover, when the determined temperature differs from the preset bathing temperature information which has been preset, recommendatory information including bathing temperature information indicating the determined temperature is generated. Consequently, a temperature of warm water to be placed in the bath is presentable so that the temperature is optimal to the current body temperature of the user.

In the information processing method, the external information may include preset bathing temperature information about a temperature of warm water to be placed in a bath tab, the temperature being preset by the user. In the generating of the recommendatory information, when the body temperature information indicates a predetermined value or lower, a specific temperature of warm water to be placed in the bath tab may be determined to be higher than the preset bathing temperature information, and the recommendatory information including bathing temperature information indicating the determined temperature may be generated.

According to this configuration, when the body temperature information about the user indicates the predetermined value or lower, a temperature of warm water to be placed in the bath tab is determined to be higher than the preset bathing temperature information which has been preset, and recommendatory information including bathing temperature information indicating the determined temperature is generated. In this regard, when the body temperature information about the user indicates the predetermined value or lower, a temperature of warm water to be placed in the bath tab is set to be higher than the current preset bathing temperature, and thus the user can warm the body thereof.

Moreover, the disclosure can be realized as: the information processing method executing the above-described distinctive ways; and an information processing device including each distinctive feature corresponding to the distinctive ways executed by the information processing method. Additionally, the disclosure can be realized by a computer program causing a computer to execute the distinctive ways included in the information processing method. From these perspectives, the same advantageous effects as those of the information processing method are achievable in the following other aspects.

An information processing device according to another aspect of the disclosure includes: a body temperature information acquisition part that acquires body temperature information indicating a body temperature of a user and associated with a user ID specifying the user; an external information acquisition part that acquires external information generated on the basis of preference information about the user; a generation part that generates, on the basis of the body temperature information and the external information, recommendatory information for the user specified by the user ID; and an output part that outputs the recommendatory information.

A non-transitory computer readable recording medium according to another aspect of the disclosure stores an information processing program, the information processing program includes: causing a computer to execute: acquiring body temperature information indicating a body temperature of a user and associated with a user ID specifying the user; acquiring external information generated on the basis of preference information about the user; generating, on the basis of the body temperature information and the external information, recommendatory information for the user specified by the user ID; and outputting the recommendatory information.

Hereinafter, embodiments of the disclosure will be described with reference to the accompanying drawings. It should be noted that each of the following embodiments illustrates one example of the disclosure, and does not delimit the technical scope of the disclosure.

### First Embodiment

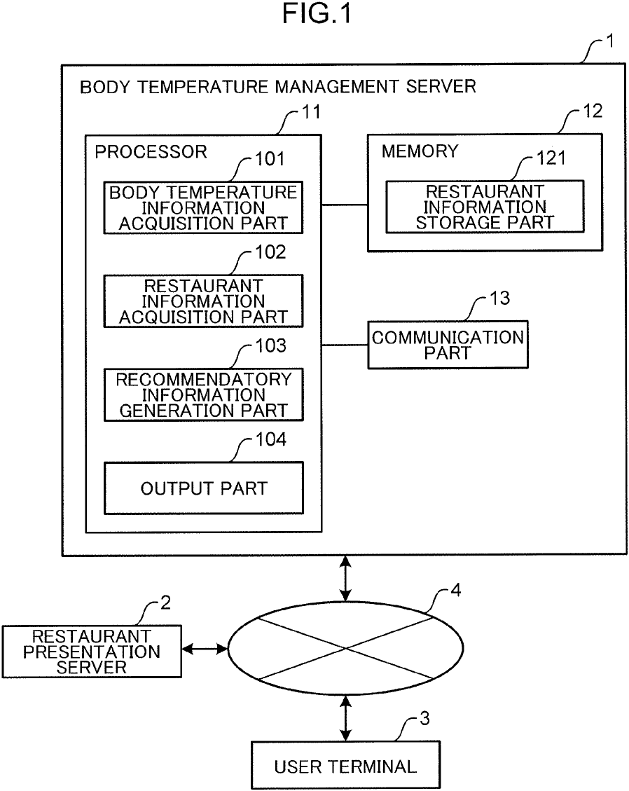

FIG. 1 is a diagram showing an example of a configuration of a body temperature management system in a first embodiment of the disclosure.

The body temperature management system shown in FIG. 1 includes a body temperature management server 1, a restaurant presentation server 2, and a user terminal 3.

The body temperature management server 1 includes, for example, a cloud server, and is communicably connected to the restaurant presentation server 2 and the user terminal 3 via a network 4. The network 4 includes, for example, the internet. A configuration of the body temperature management server 1 will be described later.

The restaurant presentation server 2 stores a plurality of pieces of restaurant information about restaurants in advance. When a restaurant search request including at least one search item is received from the user terminal 3, the restaurant presentation server 2 extracts restaurant information agreeing with the at least one search item from among the stored restaurant information, and transmits the extracted restaurant information to the user terminal 3 as a restaurant search result. The restaurant presentation server 2 may extract a piece of restaurant information, or extract a plurality of pieces of or a set of restaurant information, or one or more pieces of restaurant information instead.

Examples of the search item include a region name, a dish type, and a restaurant name. When there is no restaurant information agreeing with the at least one search item, the restaurant presentation server 2 transmits, to the user terminal 3, a restaurant search result showing no existence of restaurant information agreeing with the at least one search item.

The restaurant presentation server 2 further generates, on the basis of preference information about the user, external information. In the first embodiment, the external information includes a set of restaurant information about restaurants recommendable for the user.

The restaurant presentation server 2 receives a user ID and a restaurant search history from the user terminal 3. The restaurant search history includes at least one search item searched in past by a user identified by the user ID. The restaurant search history serves as an example of the preference information. The restaurant presentation server 2 extracts, on the basis of the received restaurant search history, a set of restaurant information recommendable for the user specified by the user ID, and transmits the extracted set of restaurant information and user ID to the body temperature management server 1.

For instance, the restaurant presentation server 2 may extract, as the set of restaurant information recommendable for the user, restaurant information agreeing with a dish type having the highest search frequency from among a plurality of dish types included in the restaurant search history. Further, for example, the restaurant presentation server 2 may extract, as the set of the restaurant information recommendable for the user, restaurant information agreeing with a region name having the highest search frequency from among a plurality of region names included in the restaurant search history.

The user terminal 3 is, for example, a smartphone, a personal computer, or a tablet-type computer, and is used by the user.

The user terminal 3 receives an input of a body temperature by the user, and transmits the received body temperature of the user and the user ID to the body temperature management server 1. The user ID is stored in the user terminal 3 in advance. The user measures a body temperature thereof by using a thermometer, and inputs the measured body temperature to the user terminal 3. When the user terminal 3 is communicable with the thermometer, the user terminal 3 may acquire the body temperature of the user from the thermometer. The user terminal 3 receives an input of a body temperature by the user, and transmits the received body temperature of the user and the user ID to the body temperature management server 1, for example, once a day.

The user terminal 3 further stores a restaurant search application for searching a desired restaurant by the user, and enables search for the desired restaurant when the restaurant search application is activated. The user terminal 3 receives an input of at least one search item by the user for searching the desired restaurant, and transmits a restaurant search request including the at least one search item to the restaurant presentation server 2. The user terminal 3 receives a restaurant search result from the restaurant presentation server 2, and displays the received restaurant search result. In this manner, the user can search the desired restaurant.

Besides, the user terminal 3 periodically transmits the user ID and the restaurant search history to the restaurant presentation server 2. The user terminal 3 transmits the user ID and the restaurant search history to the restaurant presentation server 2, for example, once a day.

Although the body temperature management system includes the single user terminal 3 in the first embodiment, the disclosure is not particularly limited thereto, and the system may include a plurality of user terminals 3.

Moreover, although the user terminal 3 transmits the user ID and the restaurant search history to the restaurant presentation server 2 in the first embodiment, the disclosure is not particularly limited thereto, and the user terminal 3 may transmit the user ID and an action history of the user to the restaurant presentation server 2. The action history represents a place which the user has visited. The restaurant presentation server 2 receives the user ID and the action history from the user terminal 3. The action history serves as an example of the preference information. The restaurant presentation server 2 may specify, on the basis of the received action history, a type of a dish providable by a restaurant having a frequency of visit by the user higher than a predetermined frequency, and may extract, as the set of restaurant information recommendable for the user, restaurant information agreeing with the specified type of the dish. The restaurant presentation server 2 may specify, on the basis of the received action history, a region having a frequency of visit by the user higher than a predetermined frequency, and may extract, as the restaurant information recommendable for the user, restaurant information about a restaurant located in the specified region.

The user terminal 3 may transmit the user ID, the action history of the user, and the restaurant search history to the restaurant presentation server 2. The restaurant presentation server 2 may generate, on the basis of the action history of the user and the restaurant search history, the set of restaurant information recommendable for the user.

The body temperature management server shown in FIG. 1 includes a processor 11, a memory 12, and a communication part 13. The body temperature management server 1 serves as an example of an information processing device.

The processor 11 includes, for example, a CPU (central processing unit). The processor 11 realizes a body temperature information acquisition part 101, a restaurant information acquisition part 102, a recommendatory information generation part 103, and an output part 104.

For instance, the memory 12 includes a storage device, such as a RAM (Random Access Memory), an HDD (Hard Disk Drive), an SSD (Solid State Drive), or a flash memory, for storing various kinds of information. The memory 12 realizes a restaurant information storage part 121.

The communication part 13 receives a user ID and restaurant information transmitted from the restaurant presentation server 2. The communication part 13 stores the received restaurant information in the restaurant information storage part 121 in association with the user ID. The communication part 13 further receives body temperature information transmitted from the user terminal 3.

The restaurant information storage part 121 stores external information generated on the basis of preference information about a user in association with the user ID. In other words, the restaurant information storage part 121 stores restaurant information about restaurants recommendable for the user in association with the user ID.

The body temperature information acquisition part 101 acquires, from the communication part 13, the body temperature information indicating the body temperature of the user and associated with the user ID specifying the user.

The restaurant information acquisition part 102 acquires the external information generated on the basis of preference information about the user. The restaurant information acquisition part 102 acquires a set of restaurant information about restaurants recommendable for the user from the restaurant information storage part 121.

The recommendatory information generation part 103 generates, on the basis of the body temperature information and the external information, recommendatory information for the user specified by the user ID. In the first embodiment, the recommendatory information generation part 103 generates, on the basis of the body temperature information and the restaurant information, recommendatory information for the user specified by the user ID.

The recommendatory information generation part 103 determines whether the body temperature information indicates a predetermined value or lower. The predetermined value is, for example, 37.0° C. defined as a determination criterion as to whether the user has a fever. When the body temperature information indicates the predetermined value or lower, the recommendatory information generation part 103 generates recommendatory information including the set of restaurant information.

The output part 104 outputs the recommendatory information generated by the recommendatory information generation part 103. The output part 104 outputs the recommendatory information to the communication part 13. The communication part 13 transmits the recommendatory information to the user terminal 3.

Next, notification of information by the body temperature management server 1 in the first embodiment of the disclosure will be described.

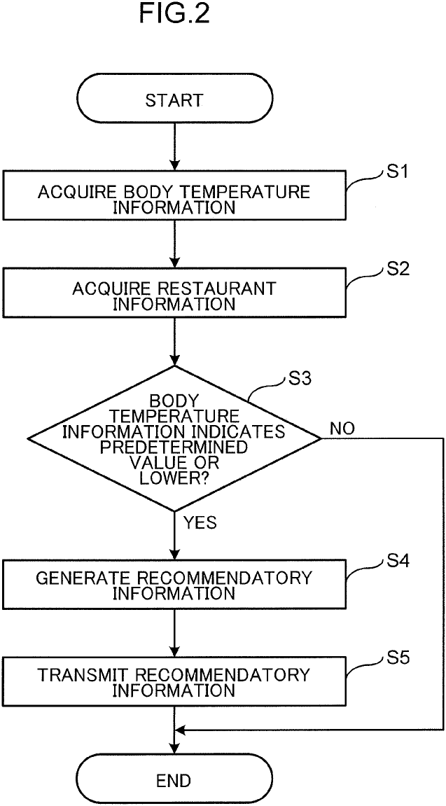

FIG. 2 is a flowchart explaining the notification of information by the body temperature management server 1 in the first embodiment of the disclosure.

First, in step S1, the body temperature information acquisition part 101 acquires, from the communication part 13, body temperature information indicating a body temperature of a user and associated with a user ID. The communication part 13 receives the body temperature information transmitted from the user terminal 3. The body temperature information acquisition part 101 acquires the body temperature information received by the communication part 13.

Next, in step S2, the restaurant information acquisition part 102 acquires a set of restaurant information about restaurants recommendable for the user and associated with the user ID from the restaurant information storage part 121. At this time, the restaurant information acquisition part 102 acquires, from the restaurant information storage part 121, a set of restaurant information corresponding to the user ID associated with the body temperature information acquired by the body temperature information acquisition part 101.

Subsequently, in step S3, the recommendatory information generation part 103 determines whether the body temperature information acquired by the body temperature information acquisition part 101 indicates a predetermined value or lower. Here, when the body temperature information is determined to exceed the predetermined value (NO in step S3), the flow finishes without generating recommendatory information.

By contrast, when the body temperature information is determined to indicate the predetermined value or lower (YES in step S3), the recommendatory information generation part 103 generates, in step S4, recommendatory information including the set of restaurant information acquired by the restaurant information acquisition part 102 in association with the user ID.

Then, in step S5, the output part 104 transmits the recommendatory information generated by the recommendatory information generation part 103 to the user terminal 3 via the communication part 13. The output part 104 transmits the recommendatory information to the user terminal 3 having transmitted the body temperature information. The user terminal 3 receives the recommendatory information. The user terminal 3 further causes a display part to display the recommendatory information.

As described heretofore, recommendatory information is generated for the user on the basis of body temperature information indicating a body temperature of the user and external information generated on the basis of preference information about the user, and the generated recommendatory information is output. This achieves provision of recommendatory information attracting the user to have an interest over an entirety of life from the body temperature information about the user.

Moreover, when the body temperature information about the user indicates a predetermined value or lower, recommendatory information including a set of restaurant information about restaurants recommendable for the user is generated. Therefore, for example, a restaurant in which the user has an interest is representable when the user has no fever.

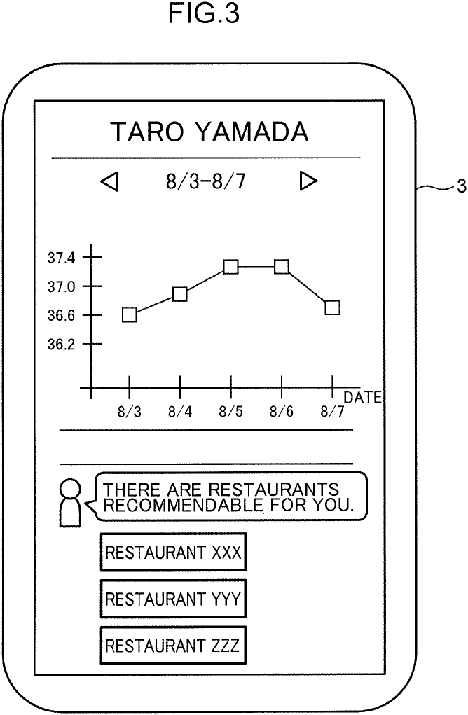

FIG. 3 shows an example of a recommendatory information presentation screen image displayed on the user terminal 3 in the first embodiment.

As shown in FIG. 3, when the body temperature information indicates a predetermined value or lower, the user terminal 3 displays a recommendatory information presentation screen image including recommendatory information about restaurants recommendable for the user. The recommendatory information presentation screen image may include a body temperature history of the user in a period of five days together with the recommendatory information. The user terminal 3 generates and displays the recommendatory information presentation screen image when receiving the recommendatory information from the body temperature management server 1.

In FIG. 3, the displayed recommendatory information presentation screen image includes: the name “Taro YAMADA” of the user; a body temperature history in a period from August 3^{rd} to August 7^{th}; and a sentence saying, “There are restaurants recommendable for you.”; and a plurality of restaurants named “restaurant XXX”, “restaurant YYY”, and “restaurant ZZZ” recommendable for the user. When the user selects one name of a specific restaurant from among the names of the restaurants, information about the restaurant corresponding to the selected name is displayed in detail, e.g., menu of dishes providable in the restaurant, the map around the restaurant, and images photographed in the restaurant.

The user can confirm, by seeing the recommendatory information presentation screen image displayed on the user terminal 3, recommendatory information (restaurant information) recommendable in accordance with the preference of the user when the body temperature thereof indicates the predetermined value or lower.

Although the body temperature history is expressed on a graph on the recommendatory information presentation screen image shown in FIG. 3, this disclosure is not particularly limited thereto, and a way of displaying the body temperature history is not limited to the aforementioned way. The recommendatory information presentation screen image may show only a body temperature on an exact date in place of the body temperature history.

### Second Embodiment

In the first embodiment, recommendatory information is generated in consideration of body temperature information. By contrast, in a second embodiment, recommendatory information is generated in consideration of a congestion density in a region where a restaurant is located and vacancy information about the restaurant in addition to body temperature information.

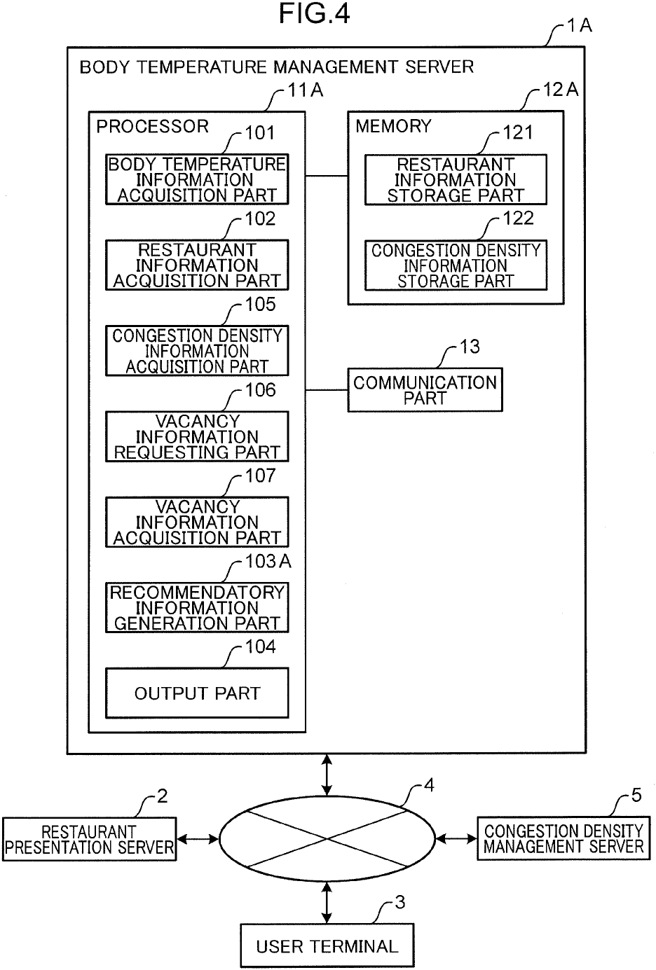

FIG. 4 is a diagram showing an example of a configuration of a body temperature management system in the second embodiment of the disclosure.

The body temperature management system shown in FIG. 4 includes a body temperature management server 1A, a restaurant presentation server 2, a user terminal 3, and a congestion density management server 5. In the second embodiment, elements which are the same as those in the first embodiment are given the same reference signs and numerals, and thus explanation therefor will be omitted.

The congestion density management server 5 generates congestion density information indicating a congestion density per region, and transmits the generated congestion density information to the body temperature management server 1A. The congestion density management server 5 generates congestion density information indicating a congestion density, for example, per nation, per prefecture, or per city, town, or village.

The congestion density is expressed at, for example, six states of level 5 to level 0. Level 5 indicates the highest congestion density. The congestion density level is determined in accordance with, for example, a population per region. For instance, a congestion density level in a region having the population of 0 to 5000 is set to level 0, a congestion density level in a region having the population of 5001 to 10000 is set to level 1, a congestion density level in a region having the population of 10001 to 15000 is set to level 2, a congestion density level in a region having the population of 15001 to 20000 is set to level 3, a congestion density level in a region having the population of 20001 to 25000 is set to level 4, and a congestion density level in a region having the population of 25000 or more is set to level 5. Each population is measured with, for example, the number of mobile phone signals received by a base station provided in each region. The above-described way of setting each congestion density level is a mere example, and thus the way of setting is not limited thereto.

The congestion density may represent a congestion rate at a station closest to a restaurant, or a congestion rate in a train which stops at the station closest to the restaurant.

The congestion density management server 5 periodically (e.g., every one hour) generates congestion density information and transmits the generated congestion density information to the body temperature management server 1A.

The body temperature management server 1A shown in FIG. 4 includes a processor 11A, a memory 12A, and a communication part 13. The body temperature management server 1A serves as an example of the information processing device.

The processor 11A realizes a body temperature information acquisition part 101, a restaurant information acquisition part 102, a recommendatory information generation part 103A, an output part 104, a congestion density information acquisition part 105, a vacancy information requesting part 106, and a vacancy information acquisition part 107. The memory 12A realizes a restaurant information storage part 121 and a congestion density information storage part 122.

The communication part 13 receives the congestion density information transmitted from the congestion density management server 5. The communication part 13 stores the received congestion density information in the congestion density information storage part 122.

The congestion density information storage part 122 stores congestion density information indicating a congestion density per region.

The congestion density information acquisition part 105 acquires congestion density information about a congestion density in a region where a restaurant recommendable for the user is located. The congestion density information acquisition part 105 acquires, from the congestion density information storage part 122, congestion density information indicating a congestion density in a region where a restaurant indicated by restaurant information acquired by the restaurant information acquisition part 102 is located.

The vacancy information requesting part 106 requests vacancy information about vacancy availability of the restaurant recommendable for the user. The vacancy information requesting part 106 transmits, to the restaurant presentation server 2 via the communication part 13, a request signal of requesting the vacancy information about the restaurant indicated by the restaurant information acquired by the restaurant information acquisition part 102. The request signal includes identification information for identifying the restaurant indicated by the restaurant information acquired by the restaurant information acquisition part 102.

The restaurant presentation server 2 manages vacancy information about each restaurant. When receiving a request signal transmitted from the body temperature management server 1A, the restaurant presentation server 2 transmits, to the body temperature management server 1A, vacancy information about a restaurant corresponding to the identification information included in the request signal. The vacancy information represents current vacancy availability of the restaurant. The communication part 13 receives the vacancy information transmitted from the restaurant presentation server 2.

The vacancy information acquisition part 107 acquires vacancy information about vacancy availability of a restaurant recommendable for the user. The vacancy information acquisition part 107 acquires the vacancy information from the communication part 13.

When the body temperature information indicates a predetermined value or lower, the recommendatory information generation part 103A selects restaurant information about a restaurant located in a region whose congestion density indicates a predetermined value or lower and having a vacancy, and generates recommendatory information including the selected restaurant information.

Next, notification of information by the body temperature management server 1A in the second embodiment of the disclosure will be described.

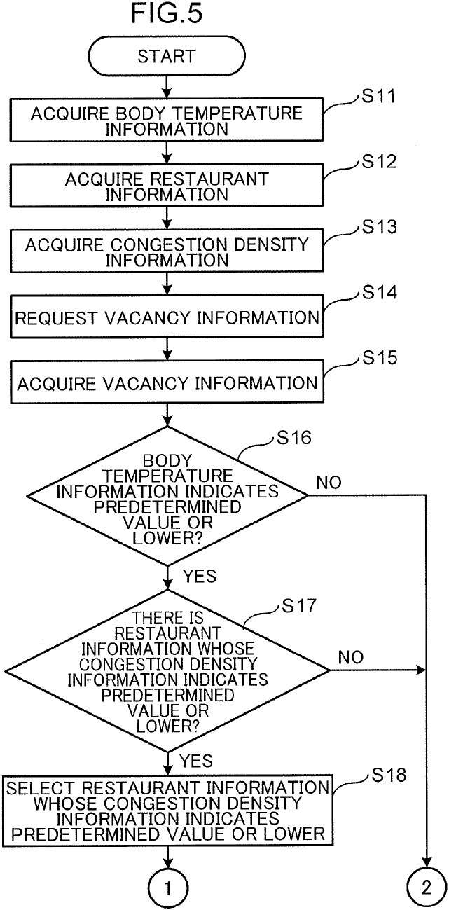

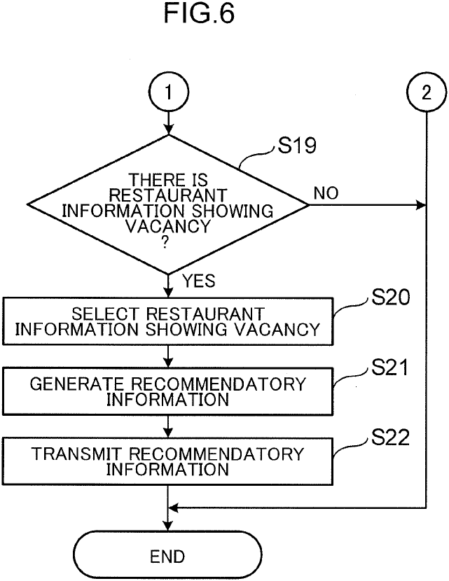

FIG. 5 is a first flowchart explaining the notification of information by the body temperature management server 1A in the second embodiment of the disclosure. FIG. 6 is a second flowchart explaining the notification of information by the body temperature management server 1A in the second embodiment of the disclosure.

Steps S11 to S12 are the same as steps S1 to S2 in FIG. 2, and thus the descriptions therefor are omitted.

Next, in step S13, the congestion density information acquisition part 105 acquires, from the congestion density information storage part 122, congestion density information indicating a congestion density in a region where a restaurant indicated by restaurant information acquired by the restaurant information acquisition part 102 is located. When a plurality of pieces of or a set of restaurant information is acquired, the congestion density information acquisition part 105 acquires, from the congestion density information storage part 122, congestion density information indicating a congestion density in a region where each of relevant restaurants is located.

Although the congestion density information storage part 122 stores the congestion density information in advance in the second embodiment, this disclosure is not particularly limited thereto. The congestion density information acquisition part 105 may demand, from the congestion density management server 5, congestion density information in a region where a restaurant indicated by the restaurant information acquired by the restaurant information acquisition part 102 is located. In this case, the congestion density information acquisition part 105 may acquire the congestion density information from the congestion density management server 5 via the communication part 13.

Subsequently, in step S14, the vacancy information requesting part 106 requests the restaurant presentation server 2 to present vacancy information indicating current vacancy availability of the restaurant indicated by the restaurant information acquired by the restaurant information acquisition part 102. The communication part 13 transmits, to the restaurant presentation server 2, a request signal of requesting the vacancy information about the restaurant indicated by the restaurant information acquired by the restaurant information acquisition part 102.

Then, in step S15, the vacancy information acquisition part 107 acquires the vacancy information indicating the current vacancy availability of the restaurant indicated by the restaurant information acquired by the restaurant information acquisition part 102. When a plurality of pieces of or a set of restaurant information is acquired, the vacancy information acquisition part 107 acquires vacancy information about each of the relevant restaurants.

Although the vacancy information acquisition part 107 acquires vacancy information indicating current vacancy availability of each restaurant recommendable for the user in the second embodiment, this disclosure is not particularly limited thereto, and the vacancy information acquisition part may acquire vacancy information indicating vacancy availability of the restaurant recommendable for the user on one day from the current time to a closing time.

Next, in step S16, the recommendatory information generation part 103A determines whether the body temperature information acquired by the body temperature information acquisition part 101 indicates the predetermined value or lower. Here, when the body temperature information is determined to exceed the predetermined value (NO in step S16), the flow finishes without generating recommendatory information.

By contrast, when the body temperature information is determined to indicate the predetermined value or lower (YES in step S16), the recommendatory information generation part 103A determines, in step S17, whether the set of restaurant information acquired by the restaurant information acquisition part 102 includes restaurant information whose congestion density information acquired by the congestion density information acquisition part 105 indicates a predetermined value or lower. For instance, the recommendatory information generation part 103A determines whether there is restaurant information having a congestion density level at level 2 or lower. Here, when it is determined that there is no restaurant information whose congestion density information indicates the predetermined value or lower (NO in step S17), the flow finishes without generating recommendatory information.

By contrast, when it is determined that there is restaurant information whose congestion density information indicates the predetermined value or lower (YES in step S17), the recommendatory information generation part 103A selects, in step S18, the restaurant information whose congestion density information indicates the predetermined value or lower from the set of restaurant information acquired by the restaurant information acquisition part 102.

Subsequently, in step S19, the recommendatory information generation part 103A determines whether the restaurant information whose selected congestion density information indicates the predetermined value or lower includes restaurant information showing a vacancy. Here, when it is determined that there is no restaurant information showing a vacancy (NO in step S19), the flow finishes without generating recommendatory information.

Contrarily, when it is determined that there is restaurant information showing a vacancy (YES in step S19), the recommendatory information generation part 103A selects, in step S20, the restaurant information showing the vacancy from the selected restaurant information whose congestion density information indicates the predetermined value or lower.

Then, in step S21, the recommendatory information generation part 103A generates recommendatory information including the restaurant information selected, from the restaurant information acquired by the restaurant information acquisition part 102, as having congestion density information indicating the predetermined value or lower, and indicating a vacancy in association with the user ID.

Step S22 is the same as step S5 in FIG. 2, and thus the descriptions therefor are omitted.

As described heretofore, when body temperature information about a user indicates a predetermined value or lower, restaurant information about a restaurant having a vacancy is selected, on the basis of vacancy information indicating vacancy availability of each restaurant recommendable for the user, from a set of restaurant information recommendable for the user, and recommendatory information including the selected restaurant information is generated. Therefore, for example, a restaurant in which the user has an interest and vacancy availability or nonavailability of the restaurant are presentable when the user has no fever.

Moreover, when the body temperature information about the user indicates the predetermined value or lower, restaurant information about a restaurant in a region whose congestion density indicates a predetermined value or lower is selected from the set of restaurant information recommendable for the user, and recommendatory information including the selected restaurant information is generated. Therefore, for example, a restaurant which is located in a region whose congestion density indicates the predetermined value or lower and in which the user has an interest is representable when the user has no fever.

Although the recommendatory information generation part 103A selects restaurant information recommendable for the user on the basis of body temperature information, congestion density information, and vacancy information in the second embodiment, this disclosure is not particularly limited thereto. The recommendatory information generation part 103A may select, on the basis of the body temperature information and the congestion density information, restaurant information recommendable for the user. In this case, when the body temperature information indicates the predetermined value or lower, the recommendatory information generation part 103A selects restaurant information about a restaurant located in a region whose congestion density indicates a predetermined value or lower, and generates recommendatory information including the selected restaurant information. Alternatively, the recommendatory information generation part 103A may select, on the basis of the body temperature information and the vacancy information, restaurant information recommendable for the user. In this case, when the body temperature information indicates the predetermined value or lower, the recommendatory information generation part 103A selects, on the basis of vacancy information, restaurant information about a restaurant having a vacancy, and generates recommendatory information including the selected restaurant information.

### Third Embodiment

In the first embodiment, when body temperature information indicates a predetermined value or lower, recommendatory information including a set of restaurant information about restaurants recommendable for a user is generated. By contrast, in a third embodiment, recommendatory information including a set of recipe information about dish recipes recommendable for a user is generated on the basis of body temperature information.

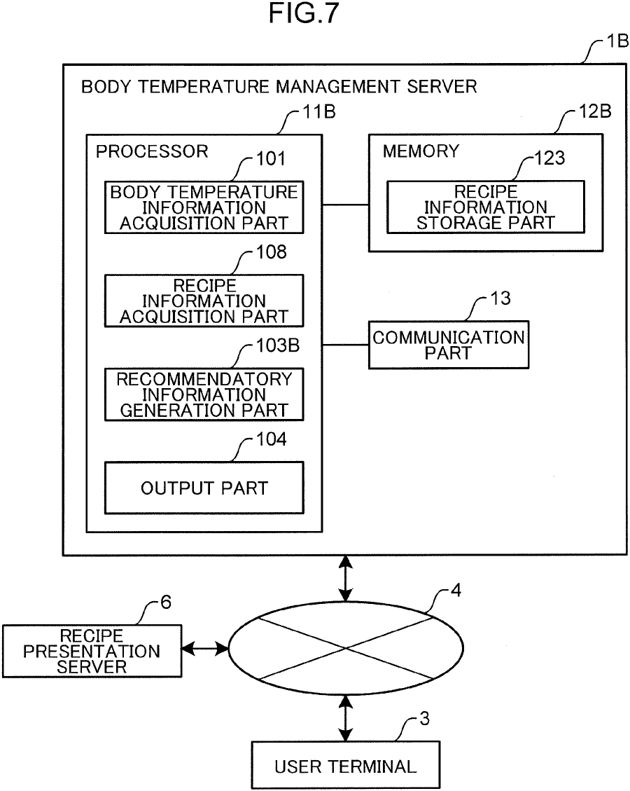

FIG. 7 is a diagram showing an example of a configuration of a body temperature management system in the third embodiment of the disclosure.

The body temperature management system shown in FIG. 7 includes a body temperature management server 1B, a user terminal 3, and a recipe presentation server 6. In the third embodiment, elements which are the same as those in the first embodiment are given the same reference signs and numerals, and thus explanation therefor will be omitted.

The recipe presentation server 6 stores a plurality of pieces of recipe information about recipes in advance. When a recipe search request including at least one search item is received from the user terminal 3, the recipe presentation server 6 extracts recipe information agreeing with the at least one search item from among the stored recipe information, and transmits the extracted recipe information to the user terminal 3 as a recipe search result. The recipe presentation server 6 may extract a piece of recipe information, or extract a plurality of pieces of or a set of recipe information, or one or more pieces of recipe information instead.

Examples of the search item include a dish type, a dish name, and a food material. When there is no recipe information agreeing with the at least one search item, the recipe presentation server 6 transmits, to the user terminal 3, a recipe search result showing no existence of recipe information agreeing with the at least one search item.

The recipe information represents a cooking way of a dish. Examples of the recipe information include a dish name, a time required for cooking, food materials necessary for cooking, an amount of each food material, a tool necessary for cooking, and a cooking procedure.

The recipe presentation server 6 further generates, on the basis of preference information about the user, external information. In the third embodiment, the external information includes a set of recipe information about dish recipes recommendable for the user.

The recipe presentation server 6 receives a user ID and a recipe search history from the user terminal 3. The recipe search history includes at least one search item searched in past by a user identified by the user ID. The recipe search history serves as an example of the preference information. The recipe presentation server 6 extracts, on the basis of the received recipe search history, a set of recipe information recommendable for the user specified by the user ID, and transmits the extracted set of recipe information and the user ID to the body temperature management server 1B.

For instance, the recipe presentation server 6 may extract, as the set of recipe information recommendable for the user, recipe information agreeing with a dish type having the highest search frequency from among a plurality of dish types included in the recipe search history. For instance, the recipe presentation server 6 may extract, as the set of recipe information recommendable for the user, recipe information agreeing with a dish name having the highest search frequency from among a plurality of dish names included in the recipe search history.

The user terminal 3 further stores a recipe search application for searching a desired recipe by the user, and achieves search for the desired recipe when the recipe search application is activated. The user terminal 3 receives an input of at least one search item by the user for searching the desired recipe, and transmits a recipe search request including the at least one search item to the recipe presentation server 6. The user terminal 3 receives a recipe search result from the recipe presentation server 6, and displays the received recipe search result. In this manner, the user can search the desired recipe.

Besides, the user terminal 3 periodically transmits the user ID and the recipe search history to the recipe presentation server 6. The user terminal 3 transmits the user ID and the recipe search history to the recipe presentation server 6, for example, once a day.

Moreover, the user terminal 3 transmits the user ID and the recipe search history to the recipe presentation server 6 in the third embodiment, this disclosure is not particularly limited thereto, and the user terminal 3 may transmit the user ID and a meal history of the user to the recipe presentation server 6. The meal history represents a dish which the user has taken. The recipe presentation server 6 receives the user ID and the meal history from the user terminal 3. The meal history serves as an example of the preference information. The recipe presentation server 6 may specify, on the basis of the received meal history, a dish type having a frequency of taking by the user higher than a predetermined frequency, and extract, as the set of recipe information recommendable for the user, recipe information agreeing with the specified dish type. The recipe presentation server 6 may specify, on the basis of the received meal history, a dish type currently taken by the user, and extract, as the set of recipe information recommendable for the user, recipe information agreeing with a dish type other than the specified dish type.

The user terminal 3 may transmit the user ID, the meal history of the user, and the recipe search history to the recipe presentation server 6. The recipe presentation server 6 may generate, on the basis of the meal history of the user and the recipe search history, a set of recipe information recommendable for the user.

The body temperature management server 1B shown in FIG. 7 includes a processor 11B, a memory 12B, and a communication part 13. The body temperature management server 1B serves as an example of the information processing device.

The processor 11B realizes a body temperature information acquisition part 101, a recommendatory information generation part 103B, an output part 104, and a recipe information acquisition part 108. The memory 12B realizes a recipe information storage part 123.

The communication part 13 receives the user ID and the recipe information transmitted from the recipe presentation server 6. The communication part 13 stores the received recipe information in the recipe information storage part 123 in association with the user ID.

The recipe information storage part 123 stores external information generated on the basis of the preference information about the user in association with the user ID. In other words, the recipe information storage part 123 stores recipe information about dish recipes recommendable for the user in association with the user ID. The recipe information includes information indicating any one of a warm dish, a cold dish, and a digestive dish.

The recipe information acquisition part 108 acquires the external information generated on the basis of preference information about the user. The recipe information acquisition part 108 acquires a set of recipe information about dish recipes recommendable for the user from the recipe information storage part 123.

The recommendatory information generation part 103B generates, on the basis of the body temperature information and the external information, recommendatory information for the user specified by the user ID. In the third embodiment, the recommendatory information generation part 103B generates, on the basis of the body temperature information and the recipe information, recommendatory information for the user specified by the user ID.

The recommendatory information generation part 103B selects, on the basis of the body temperature information, recipe information, and generates recommendatory information including the selected recipe information. The recommendatory information generation part 103B determines whether the body temperature information indicates a predetermined value or lower. The predetermined value is, for example, 37.0° C. defined as a determination criterion as to whether the user has a fever. The recommendatory information generation part 103B generates recommendatory information including recipe information about a warm dish when the body temperature information indicates the predetermined value or lower. The recommendatory information generation part 103B generates recommendatory information including recipe information about a cold dish or a digestive dish when the body temperature information exceeds the predetermined value.

Next, notification of information by the body temperature management server 1B in the third embodiment of the disclosure will be described.

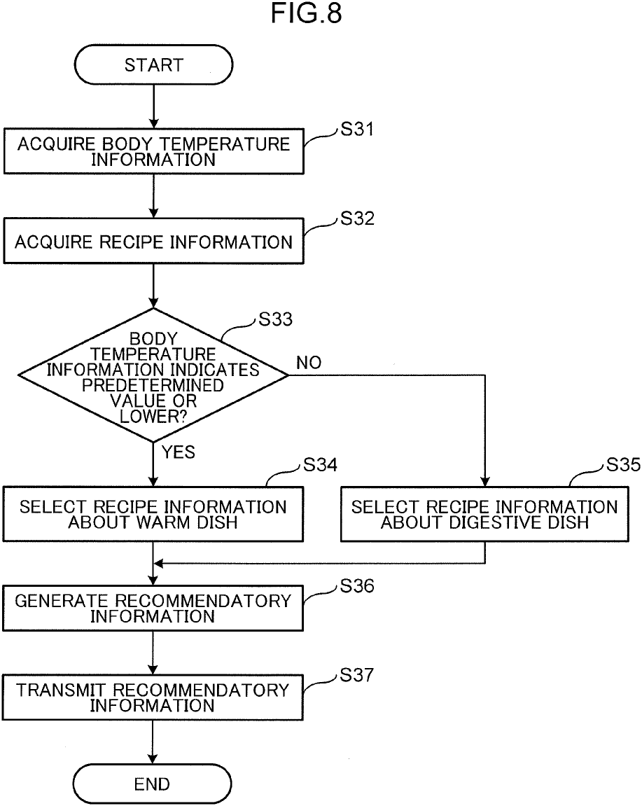

FIG. 8 is a flowchart explaining the notification of information by the body temperature management server 1B in the third embodiment of the disclosure.

Step S31 is the same as step S1 in FIG. 2, and thus the descriptions therefor are omitted.

Subsequently, in step S32, the recipe information acquisition part 108 acquires a set of recipe information about dish recipes recommendable for a user and associated with a user ID from the recipe information storage part 123. At this time, the recipe information acquisition part 108 acquires, from the recipe information storage part 123, a set of recipe information corresponding to the user ID associated with body temperature information acquired by the body temperature information acquisition part 101.

Subsequently, in step S33, the recommendatory information generation part 103B determines whether the body temperature information acquired by the body temperature information acquisition part 101 indicates a predetermined value or lower. Here, when the body temperature information is determined to indicate the predetermined value or lower (YES in step S33), the recommendatory information generation part 103B selects, in step S34, recipe information about a warm dish from the set of recipe information acquired by the recipe information acquisition part 108.

By contrast, when the body temperature information is determined to exceed the predetermined value (No in step S33), the recommendatory information generation part 103B selects, in step S35, recipe information about a digestive dish from the set of recipe information acquired by the recipe information acquisition part 108.

Then, in step S36, the recommendatory information generation part 103B generates, in association with the user ID, recommendatory information including the recipe information about the warm dish or the recipe information about the digestive dish selected from the set of recipe information acquired by the recipe information acquisition part 108.

Further, when the body temperature information is determined to exceed the predetermined value, the recommendatory information generation part 103B may select recipe information about a cold dish from the set of recipe information acquired from the recipe information acquisition part 108.

Step S37 is the same as step S5 in FIG. 2, and thus the descriptions therefor are omitted.

As described heretofore, recipe information is selected, on the basis of body temperature information, from a set of recipe information recommendable for a user, and recommendatory information including the selected recipe information is generated. Consequently, a dish recipe which is optimal to a current body temperature of the user and attractive to the user to have an interest is presentable.

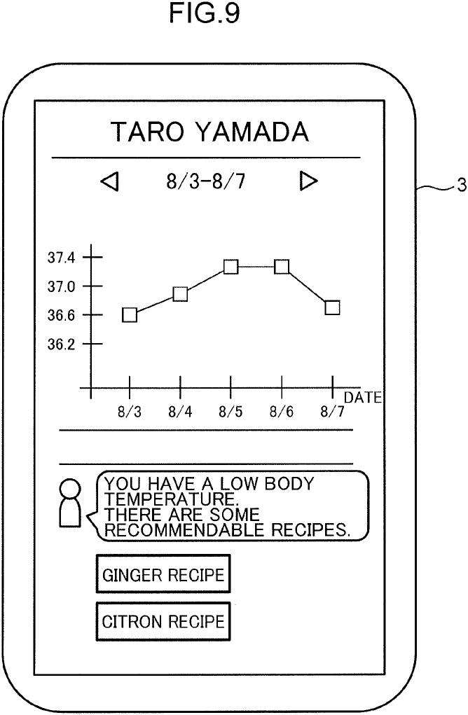

FIG. 9 shows an example of a recommendatory information presentation screen image displayed on the user terminal 3 in the third embodiment.

As shown in FIG. 9, when the body temperature information indicates a predetermined value or lower, the user terminal 3 displays a recommendatory information presentation screen image including recommendatory information about dish recipes recommendable for a user. The recommendatory information presentation screen image may include a body temperature history of the user in a period of five days together with the recommendatory information. The user terminal 3 generates and displays the recommendatory information presentation screen image when receiving the recommendatory information from the body temperature management server 1B.

In FIG. 9, the displayed recommendatory information presentation screen image includes: the name “Taro YAMADA” of the user; a body temperature history in a period from August 3^{rd} to August 7^{th}; and a set of sentences saying, “You have a low body temperature. There are some recommendable recipes.”; and a plurality of recipes named “ginger recipe” and “citron recipe” recommendable for the user. When the user selects one name of a specific recipe from among the names of the recipes, information about the recipe corresponding to the selected name is displayed in detail, e.g., a dish name, a time required for cooking, food materials necessary for cooking, an amount of each food material, a tool necessary for cooking, and a cooking procedure.

The user can confirm recommendatory information (recipe information) recommendable in accordance with a body temperature and the preference of the user by seeing the recommendatory information presentation screen image displayed on the user terminal 3.

Although the recommendatory information generation part 103B selects recipe information about a warm dish from a set of recipe information acquired by the recipe information acquisition part 108 when body temperature information indicates a predetermined value or lower, and selects recipe information about a cold dish or a digestive dish from the set of recipe information acquired by the recipe information acquisition part 108 when the body temperature information exceeds the predetermined value in the third embodiment, this disclosure is not particularly limited thereto. The set of recipe information may include a body temperature suitable for taking a dish cooked on the basis of a recipe. The recommendatory information generation part 103B may select recipe information, in accordance with body temperature information acquired by the body temperature information acquisition part 101, from the set of recipe information acquired by the recipe information acquisition part 108.

### Fourth Embodiment

In the first embodiment, when body temperature information indicates a predetermined value or lower, recommendatory information including a set of restaurant information about restaurants recommendable for a user is generated. By contrast, in a fourth embodiment, recommendatory information including bathing temperature information recommendable for a user is generated on the basis of body temperature information.

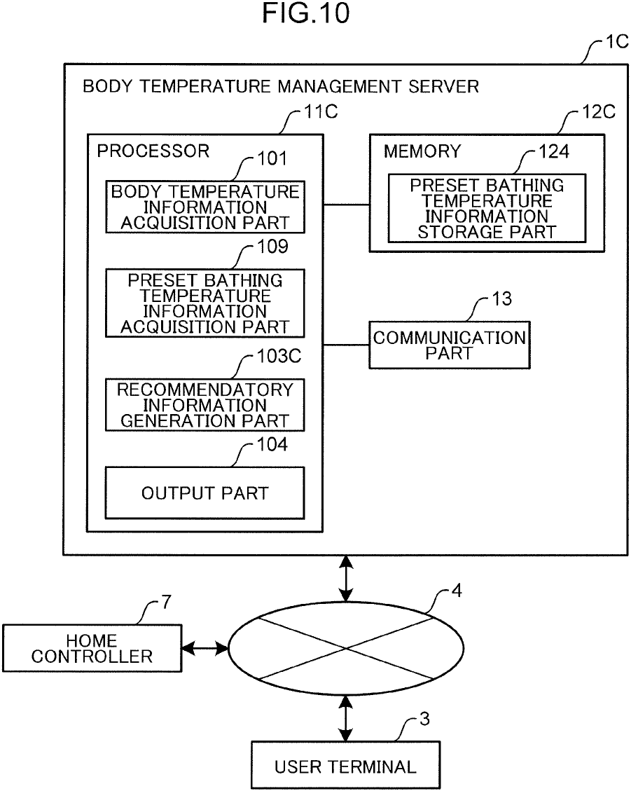

FIG. 10 is a diagram showing an example of a configuration of a body temperature management system in the fourth embodiment of the disclosure.

The body temperature management system shown in FIG. 10 includes a body temperature management server 1C, a user terminal 3, and a home controller 7. In the fourth embodiment, elements which are the same as those in the first embodiment are given the same reference signs and numerals, and thus explanation therefor will be omitted.

The home controller 7 is arranged in a house of the user and receives a manipulation by the user to home appliances and facility appliances in the house. The home controller 7 receives, from the user, a setting of a temperature of warm water to be placed in a bath tab. The user sets the temperature of warm water to be placed in the bath tab by using the home controller 7.

The home controller 7 generates, on the basis of preference information about the user, external information. In the fourth embodiment, the external information includes preset bathing temperature information about a temperature of warm water to be placed in the bath tab, the temperature being preset by the user. The temperature of the warm water preferable to the user serves as an example of the preference information. The home controller 7 transmits preset bathing temperature information and a user ID to the body temperature management server 1C, the preset bathing temperature information indicating a temperature of warm water to be placed in the bath tab, the temperature being preset by the user.

The home controller 7 periodically transmits the preset bathing temperature information and the user ID to the body temperature management server 1C. The home controller 7 transmits the preset bathing temperature information and the user ID to the body temperature management server 1C, for example, once a day. Besides, the home controller 7 may transmit the preset bathing temperature information and the user ID to the body temperature management server 1C at each updating of the preset bathing temperature information.

In place of the home controller 7, a water heater may transmit the preset bathing temperature information and the user ID to the body temperature management server 1C. Alternatively, when the user terminal 3 can set a temperature of warm water to be placed in the bath tab, the user terminal 3 may transmit the preset bathing temperature information and the user ID to the body temperature management server 1C.

The body temperature management server 1C shown in FIG. 10 includes a processor 11C, a memory 12C, and a communication part 13. The body temperature management server 1C serves as an example of the information processing device.

The processor 11C realizes a body temperature information acquisition part 101, a recommendatory information generation part 103C, an output part 104, and a preset bathing temperature information acquisition part 109. The memory 12C realizes a preset bathing temperature information storage part 124.

The communication part 13 receives the user ID and the preset bathing temperature information transmitted from the home controller 7. The communication part 13 stores the received preset bathing temperature information in the preset bathing temperature information storage part 124 in association with the user ID.

The preset bathing temperature information storage part 124 stores the external information generated on the basis of the preference information about the user in association with the user ID. Specifically, the preset bathing temperature information storage part 124 stores the preset bathing temperature information in association with the user ID.

The preset bathing temperature information acquisition part 109 acquires external information generated on the basis of preference information about the user. The preset bathing temperature information acquisition part 109 acquires, from the preset bathing temperature information storage part 124, preset bathing temperature information indicating a temperature of warm water to be placed in a bath tab, the temperature being preset by the user.

The recommendatory information generation part 103C generates, on the basis of the body temperature information and the external information, recommendatory information for the user specified by the user ID. In the fourth embodiment, the recommendatory information generation part 103C generates, on the basis of the body temperature information and the preset bathing temperature information, recommendatory information for the user specified by the user ID.

The recommendatory information generation part 103C determines whether the body temperature information indicates a predetermined value or lower. The predetermined value is, for example, 37.0° C. defined as a determination criterion as to whether the user has a fever. When the body temperature information indicates the predetermined value or lower, the recommendatory information generation part 103C determines a specific temperature of warm water to be placed in the bath tab to be higher than the preset bathing temperature information, and generates recommendatory information including bathing temperature information indicating the determined temperature.

Next, notification of information by the body temperature management server 1C in the fourth embodiment of the disclosure will be described.

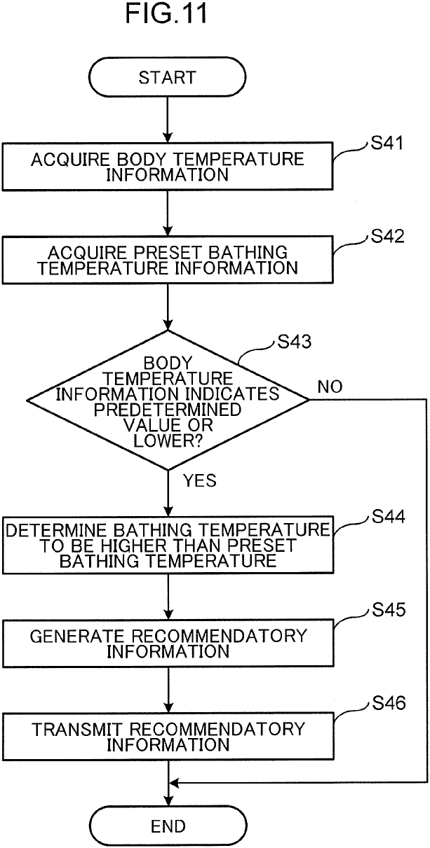

FIG. 11 is a flowchart explaining the notification of information by the body temperature management server 1C in the fourth embodiment of the disclosure.

Step S41 is the same as step S1 in FIG. 2, and thus the descriptions therefor are omitted.

Next, in step 42, the preset bathing temperature information acquisition part 109 acquires, from the preset bathing temperature information storage part 124, preset bathing temperature information associated with a user ID. At this time, the preset bathing temperature information acquisition part 109 acquires, from the preset bathing temperature information storage part 124, preset bathing temperature information corresponding to the user ID associated with body temperature information acquired by the body temperature information acquisition part 101.

Subsequently, in step S43, the recommendatory information generation part 103C determines whether the body temperature information acquired by the body temperature information acquisition part 101 indicates a predetermined value or lower. Here, when the body temperature information is determined to exceed the predetermined value (NO in step S43), the flow finishes without generating recommendatory information.

By contrast, when the body temperature information is determined to indicate the predetermined value or lower (YES in step S43), the recommendatory information generation part 103C determines, in step S44, a bathing temperature higher than the preset bathing temperature information acquired by the preset bathing temperature information acquisition part 109. For instance, the recommendatory information generation part 103C determines a bathing temperature by adding 1° C. to the preset bathing temperature.

Here, a temperature value to be added to the preset bathing temperature is not limited to 1° C. The recommendatory information generation part 103C may determine a temperature value to be added to the preset bathing temperature in accordance with a difference between the body temperature information and the predetermined period. Specifically, the recommendatory information generation part 103C may increase the temperature value to be added to the preset bathing temperature as the difference between the body temperature information and the predetermined value becomes larger.

Then, in step S45, the recommendatory information generation part 103C generates, in association with the user ID, recommendatory information including bathing temperature information indicating the determined bathing temperature.

Step S46 is the same as step S5 in FIG. 2, and thus the descriptions therefor are omitted.

As described heretofore, when body temperature information about a user indicates a predetermined value or lower, a temperature of warm water to be placed in a bath tab is determined to be higher than a preset bathing temperature information which has been preset, and recommendatory information including bathing temperature information indicating the determined temperature is generated. In this regard, when the body temperature information about the user indicates the predetermined value or lower, a temperature of warm water to be placed in the bath tab is set to be higher than the current preset bathing temperature, and thus the user can warm the body thereof.

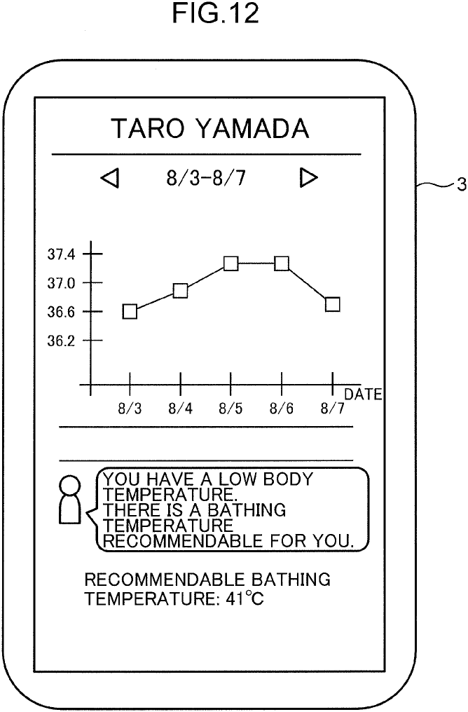

FIG. 12 shows an example of a recommendatory information presentation screen image displayed on the user terminal 3 in the fourth embodiment.

As shown in FIG. 12, when body temperature information indicates a predetermined value or lower, the user terminal 3 displays a recommendatory information presentation screen image including recommendatory information about a bathing temperature recommendable for a user. The recommendatory information presentation screen image may include a body temperature history of the user in a period of five days together with the recommendatory information. The user terminal 3 generates and displays the recommendatory information presentation screen image when receiving the recommendatory information from the body temperature management server 1C.

In FIG. 12, the displayed recommendatory information presentation screen image includes: the name “Taro YAMADA” of the user; a body temperature history in a period from August 3^{rd} to August 7^{th}; and a set of sentences saying, “You have a low body temperature. There is a bathing temperature recommendable for you.”; and the bathing temperature of “41° C.” recommendable for the user.

The user can confirm recommendatory information (bathing temperature information) recommendable in accordance with a body temperature and the preference of the user by seeing the recommendatory information presentation screen image displayed on the user terminal 3.

Although the recommendatory information generation part 103C determines a bathing temperature to be higher than the preset bathing temperature acquired by the preset bathing temperature information acquisition part 109 when the body temperature information indicates the predetermined value or lower in the fourth embodiment, this disclosure is not particularly limited thereto. The memory 12C may store a table associating a body temperature and a bathing temperature with each other in advance. The recommendatory information generation part 103C may determine, on the basis of body temperature information, a bathing temperature of warm water to be placed in the bath tab. Besides, when the determined bathing temperature differs from the preset bathing temperature information, the recommendatory information generation part 103C may generate recommendatory information including bathing temperature information indicating the determined bathing temperature.

In this case, a specific temperature of warm water to be placed in the bath tab is determined on the basis of the body temperature of the user. Moreover, when the determined temperature differs from the preset bathing temperature information which has been preset, recommendatory information including bathing temperature information indicating the determined temperature is generated. Consequently, a temperature of warm water to be placed in the bath is presentable so that the temperature is optimal to the current body temperature of the user.

Furthermore, when the determined bathing temperature differs from the preset bathing temperature, the output part 104 may transmit, to the home controller 7, bathing temperature information indicating the determined bathing temperature. The home controller 7 having received the bathing temperature information may change a current preset bathing temperature to a received bathing temperature. In this manner, the bathing temperature is automatically changeable.

A body temperature management server may generate recommendatory information in accordance with location information about a user. For instance, when temperature measurement is executed in a group on an event site, the body temperature management server can transmit local information about restaurants, hotels, and activities around the site only to each user terminal of the group. When a body temperature exceeds a threshold, the body temperature management server may transmit, to the user terminal, information about a hospital or clinic, or a health care center located near the user having the temperature exceeding the threshold.

When the body temperature management server does not receive a measured body temperature from a user terminal and receives a measured body temperature from another user terminal of another member in a group to which the user belongs, the measurement of the body temperature by another member may be notified to the user terminal. When the body temperature management server does not receive a measured body temperature from the user terminal of the user and a body temperature of another member in a group to which the user belongs is continuously measured throughout a predetermined period, the continuous measurement of the body temperature by another member throughout the predetermined period may be notified to the user terminal.

The body temperature management server may calculate a temperature measurement rate of the group to which the user belongs on an exact day, and transmits the calculated temperature measurement rate to the user terminal.

When the body temperature management server does not receive a measured body temperature from the user terminal and receives a measured temperature from a user terminal of another user whose action pattern of temperature measurement is similar to an action pattern of temperature measurement of the user, the measurement of the body temperature by another user whose action pattern of temperature measurement is similar to the action pattern of temperature measurement of the user may be notified to the user terminal. Such another user whose action pattern of temperature measurement is similar to the action pattern of temperature measurement of the user represents, for example, another user whose temperature measurement rate in a predetermined period is similar to a temperature measurement rate of the user in the period, or another user whose temperature measurement time is similar to a temperature measurement time of the user.

When the body temperature management server does not receive a measured body temperature from the user terminal and a manager of a group to which the first user belongs confirms body temperature information about a certain member in the group, confirmation of the body temperature information about the member by the manager may be notified to the user terminal.

The body temperature management server may notify the user terminal of an optimal temperature measurement place for the user and/or an optimal temperature measurement time for the user. For instance, the body temperature management server may detect an action pattern of the user and give notification of a place where the user stays longest in a period from waking up to going out as the optimal temperature measurement place for the user. For instance, the body temperature management server may give notification of a time period during which the user stays in the optimal temperature measurement place for the user as the optimal temperature measurement time for the user.

The body temperature management server may transmit alert information to the user terminal 3 when body temperature information indicating a predetermined value or higher is continuously measured throughout a predetermined period. The predetermined value is, for example, 37.5° C., and the predetermined period includes, for example, four days.

The body temperature management server may determine whether the user has a fever, and transmit, to the user terminal 3, a message encouraging self-quarantine when the user has a fever. The body temperature management server may determine that the user has a fever when the body temperature information about the user indicates 37° C. or higher.

The body temperature management server may store schedule information about a plurality of users working at the same company. When body temperature information about one user indicates 37° C. or higher among the body temperature information about the users, the body temperature management server may transmit alert information to a user terminal 3 of each user, other than the one user, who is to report to work.

The body temperature management server may change a frequency of giving notification of a message encouraging measurement of a body temperature to the user in accordance with implementation or non-implementation of a preventive measure against a virus spread (e.g., issuance or non-issuance of a declaration of a state of emergency from a government). Specifically, the body temperature management server may set a frequency of notification of a message encouraging measurement of a body temperature to the user in implementation of a preventive measure against a virus spread to be higher than a frequency of the notification of the message encouraging measurement of a body temperature to the user in non-implementation of the preventive measure against the virus spread.

The body temperature management server may change wording in a message encouraging measurement of a body temperature for the notification to the user who has not measured a body temperature thereof in accordance with implementation or non-implementation of a preventive measure against a virus spread (e.g., issuance or non-issuance of a declaration of a state of emergency from a government). Specifically, the body temperature management server may notify the user of a message saying, “Make sure to measure your body temperature.”, in implementation of a preventive measure against a virus spread, and may notify the user of a message saying, “Measure your body temperature.”, in non-implementation of the preventive measure against the virus spread.

The body temperature management system may further include a Web meeting server communicably connecting a plurality of user terminals to one another via a network to hold a Web meeting by sharing images or pictures and voices acquired at the respective terminals. Each user terminal may receive an input of a body temperature of each user and a current health condition of each user, and transmit the input body temperature information and health condition information to the body temperature management server. The body temperature management server may transmit the body temperature information and the health condition information received from each user terminal to the Web meeting server. When the Web meeting is held among a plurality of users, the Web meeting server may display, on a Web meeting screen image, an icon indicating a body temperature having a predetermined value or higher about a certain user or an icon indicating a symptom of a specific disease from a health condition of the user together with an image of the user. For instance, when the user has the body temperature of 37° C. or higher, the Web meeting server may display, on the Web meeting screen image, an icon indicating that the user has a fever together with an image of the user. Besides, for instance, when the health condition of the user indicates coughing, the Web meeting server may display, on the Web meeting screen image, an icon indicating the coughing of the user together with an image of the user. In this manner, each user can know a body temperature and a health condition of other user in the Web meeting.

In addition, the user terminal may receive an input of a residence of the user and a current health condition of the user, and transmit the input residence information and health condition information to the body temperature management server. When the health condition information about the user shows a symptom of a specific disease, the body temperature management server may specify a hospital or clinic suitable for treatment of the specific disease, and notify the user terminal of the specified hospital or clinic.

In the embodiments, each constituent element may be realized with dedicated hardware or by executing a software program suitable for the constituent element. Each constituent element may be realized by a program execution unit, such as a CPU or a processor, reading out and executing a software program recorded on a recording medium, such as a hard disk or a semiconductor memory. Other independent computer system may implement a program by recording the program in a recording medium to be transferred, or transferring the program via a network.

A part or all of functions of the device according to the embodiment of the disclosure are typically realized as a large scale integration (LSI), which is an integrated circuit. These functions may be formed as separate chips, or some or all of the functions may be included in one chip. The circuit integration is not limited to the LSI, and may be realized with a dedicated circuit or a general-purpose processor. A field programmable gate array (FPGA) that is programmable after manufacturing of an LSI or a reconfigurable processor in which connections and settings of circuit cells within the LSI are reconfigurable may be used.

A part or all of functions of the device according to the embodiment of the present disclosure may be implemented by a processor, such as a CPU executing a program.

Numerical values used above are merely illustrative to be used to specifically describe the present disclosure, and thus the present disclosure is not limited to the illustrative numerical values.

Order in which steps shown in the flowcharts are executed is merely illustrative to be used to specifically describe the present disclosure, and thus steps may be executed in order other than the above order as long as similar effects are obtained. Some of the steps may be executed simultaneously (in parallel) with other steps.

### INDUSTRIAL APPLICABILITY

The technology according to this disclosure achieves presentation of recommendatory information attracting a user to have an interest over an entirety of life from body temperature information about the user, and thus is useful as a technology of generating, on the basis of the body temperature information about the user, recommendatory information recommendable for the user.

1. An information processing method, by a computer, comprising:
acquiring body temperature information indicating a body temperature of a user and associated with a user ID specifying the user;
acquiring external information generated on the basis of preference information about the user;
generating, on the basis of the body temperature information and the external information, recommendatory information for the user specified by the user ID; and
outputting the recommendatory information.

2. The information processing method according to claim 1, wherein the external information includes a set of restaurant information about restaurants recommendable for the user, and,
in the generating of the recommendatory information, when the body temperature information indicates a predetermined value or lower, the recommendatory information including the set of restaurant information is generated.

3. The information processing method according to claim 2, further comprising acquiring vacancy information about vacancy availability of the restaurants recommendable for the user, wherein,
in the generating of the recommendatory information, when the body temperature information indicates the predetermined value or lower, restaurant information about a restaurant having a vacancy is selected, on the basis of the vacancy information, from the set of restaurant information, and the recommendatory information including the selected restaurant information is generated.

4. The information processing method according to claim 2, further comprising acquiring congestion density information about a congestion density in a region where each of the restaurants recommendable for the user is located, wherein,
in the generating of the recommendatory information, when the body temperature information indicates the predetermined value or lower, restaurant information about a restaurant located in a region whose congestion density indicates a predetermined value or smaller is selected from the set of restaurant information, and the recommendatory information including the selected restaurant information is generated.

5. The information processing method according to claim 1, wherein the external information includes a set of recipe information about dish recipes recommendable for the user, and,
in the generating of the recommendatory information, recipe information is selected, on the basis of the body temperature information, from the set of recipe information, and the recommendatory information including the selected recipe information is generated.

6. The information processing method according to claim 1, wherein the external information includes preset bathing temperature information about a temperature of warm water to be placed in a bath tab, the temperature being preset by the user, and,
in the generating of the recommendatory information, a specific temperature of warm water to be placed in the bath tab is determined on the basis of the body temperature information, and the recommendatory information including bathing temperature information indicating the determined temperature is generated when the determined temperature differs from the preset bathing temperature information.

7. The information processing method according to claim 1, wherein the external information includes preset bathing temperature information about a temperature of warm water to be placed in a bath tab, the temperature being preset by the user, and,
in the generating of the recommendatory information, when the body temperature information indicates a predetermined value or lower, a specific temperature of warm water to be placed in the bath tab is determined to be higher than the preset bathing temperature information, and the recommendatory information including bathing temperature information indicating the determined temperature is generated.

8. An information processing device, comprising:
a body temperature information acquisition part that acquires body temperature information indicating a body temperature of a user and associated with a user ID specifying the user;
an external information acquisition part that acquires external information generated on the basis of preference information about the user;
a generation part that generates, on the basis of the body temperature information and the external information, recommendatory information for the user specified by the user ID; and
an output part that outputs the recommendatory information.

9. A non-transitory computer readable recording medium storing an information processing program comprising:
causing a computer to execute:
acquiring body temperature information indicating a body temperature of a user and associated with a user ID specifying the user;
acquiring external information generated on the basis of preference information about the user;
generating, on the basis of the body temperature information and the external information, recommendatory information for the user specified by the user ID; and
outputting the recommendatory information.
